(19) United States
(12) Patent Application Publication
(10) Pub. No.: US 2012/0157318 A1
(43) Pub. Date: Jun. 21, 2012

(54) Use of Branched Copolymers in Polymer Blends

(75) Inventors: Paul Hugh Findlay, Liverpool (GB); Brodyck James Lachlan Royles, Liverpool (GB); Roselyne Marie Andree Baudry, Liverpool (GB); Neil John Simpson, Liverpool (GB); Sharon Todd, Liverpool (GB); Steven Paul Rannard, Liverpool (GB)

(73) Assignee: UNILEVER PLC, London (GB)

(21) Appl. No.: 13/394,110

(22) PCT Filed: Sep. 8, 2010
(86) PCT No.: PCT/EP2010/005502

§ 371 Date: Mar. 2, 2012

(30) Foreign Application Priority Data
Sep. 8, 2009 (GB) 0915683.7

(51) Int. Cl. A01N 25/02; C08L 33/02; C08L 33/06; C09D 133/06; C09D 125/04; C09D 181/00; C09D 11/00; C09J 133/06; C09J 125/04; C09J 181/00; C10M 145/14; C09K 8/60; A61K 47/32; A61K 8/81; C08L 81/00

(52) U.S. Cl. 504/362; 525/189; 525/451; 524/502; 524/539; 508/469; 508/473; 507/219; 507/224; 514/772.4; 514/772.6

(57) ABSTRACT

The present invention relates to the use of a branched addition copolymer in combination with a polymer in a solution or melt formulation to reduce the viscosity of the solution formulation and/or melt formulation compared to the viscosity of a solution and/or melt comprising the polymer alone wherein the branched addition copolymer is obtainable by an addition polymerisation process, methods for the preparation of the formulations, and novel branched addition copolymers for use as same.

### TECHNICAL FIELD

The present invention relates to the use of branched addition copolymers in a solution or melt formulation in combination with a polymer to reduce the viscosity of the solution formulation and/or melt formulation compared to the viscosity of the solution and/or melt comprising the polymer alone methods for the preparation of the formulations, blends comprising the branched addition copolymers and the linear analogues and novel branched addition copolymers for use in same.

That is, in addition, the present invention also relates to formulations or blends comprising at least one branched addition copolymer which is used as a replacement for a polymer component in a formulation which results in a formulation or blend of reduced solution or melt viscosity when compared to a formulation without the presence of a branched addition copolymer, preparation of the formulations and the use of such formulations.

The formulation or blend may comprise linear and branched polymers and the formulation may comprise a solution and/or melt formed from a combination of the branched and linear polymers.

In addition the present invention relates to the use of branched addition copolymers in combination with a linear analogue to reduce the elastic behaviour of a blend in a solution or melt formulation, when compared with the values for a linear polymeric material when used alone.

### BACKGROUND OF THE INVENTION

### Polymer Rheology

Viscosity is defined as the resistance of a fluid to deformation under an external stress. The viscosity of a solution is governed by the internal structure of the pure liquid or by the nature of the material dissolved or dispersed in the liquid phase. Polymeric materials dissolved in a solvent or alternatively in a molten form, typically demonstrate high viscosity values. In many cases this is advantageous to a system in which the polymeric material is placed and indeed it is commonplace for a polymer to be designed to act in just such a way and to cause an increase in viscosity, such as in the use of polymeric thickeners in many applications.

In several applications however, this high solution or melt viscosity is not desirable as it renders the formulation intractable or at the very least difficult to process or utilise in final form. In such situations it is necessary for the formulation to be either diluted with further solvent or used at a higher temperature to give a formulation of the required viscosity. An example of such a situation is in the field of coatings where the viscosity of the final coating formulation is crucial for efficient application and coverage of the coating onto the substrate. In such applications, a large amount of solvent is often required to give a workable solution. Unfortunately, where the solvent in question is a volatile organic compound (VOC) the use thereof can lead to encroachment upon environmental legislation.

In melt processing, the high viscosity of high molecular weight polymers can lead to processing difficulties with the result that high temperatures are required. The use of high temperatures in processing thus leads to high energy requirements. In many applications however, polymers are required to possess high molecular weights in order to give suitable final properties, with the result that the polymers give rise to extremely high solution or melt viscosities, which as discussed above can be problematic.

Polymeric solutions or melts also exhibit elastic behaviour and deformation under external stress. That is, polymers in a solution or melt exhibit chain entanglement which manifests itself in an elastic behaviour of the polymer solution or melt. This effect is particularly noticeable in high molecular weight linear systems where the increased levels of chain entanglements often results in the creation of highly elastic solutions or melts. In many applications this is not advantageous as it can render the melt or solution intractable and difficult to process. This elastic or “stringiness” in a formulation can also limit the amount of polymer that can be incorporated, the molecular weight of the polymer. In many areas the elastic behaviour of a formulation can affect the mode of application, this is particularly true when spraying, injecting, jetting or roller applying the formulation.

It has now surprisingly been found that branched addition co-polymeric structures do not exhibit this effect to such an extent as linear materials of equivalent molecular weight. Therefore also in accordance with the present invention there is described the use of branched addition copolymers as a replacement for linear analogues to reduce the elastic behaviour, of a melt or solution compared to the elastic characteristics of a linear polymeric material when used alone.

By the term ‘equivalent analogous linear polymer’ is meant a polymer of identical or similar chemical composition or molecular weight. For example a branched copolymer containing 70 weight percent styrene component and a linear polymer containing a 70 percent styrene component of equivalent molecular weight.

The term ‘melt’ and ‘solution’ used herein to describe the polymer relate to a formulation where the polymer is molten or softened via heat or is dissolved in a suitable solvent respectively.

The term polymer blend is used herein to define a mixture or two or more polymers in a solution or melt formulation where the polymers are thoroughly mixed within the formulation.

Whilst not wishing to be bound by any particular theory, it is thought that polymer viscosity is primarily due to the entanglement of the polymer chains in a system. Where the molecular weight or chain length reaches a critical point molecular weight, M_{c}, there is a sharp increase in viscosity. In addition, where the polymers posses inter or intra-molecular associating moieties, such as H-bond donor or accepting units, the M_{c} can be quite small leading to extremely viscous solutions or melts, such as in the case of many natural or functionalised polysaccharides or synthetic water-soluble macromolecules.

Linear polymers are commonly used in many applications due to their high solubility and ease of preparation. However, due to the architecture of these copolymers, the copolymers often give rise to high viscosity solutions or melts. In addition such linear polymers can be extremely slow or difficult to dissolve or melt in order to achieve isotropic liquids.

Viscosity lowering additives such as low molecular weight oligomers or co-solvents have been utilised to reduce the solution or melt viscosity of a higher molecular weight polymer formulation. These materials essentially plasticise the bulk polymer reducing inter and intra association leading to lower viscosities. In some occasions the use of these types of additives is undesirable as it can affect the final properties of the polymer such as leading to poor adhesion or film formation.

There has been a considerable amount of recent work undertaken to try and address the problematic issues alluded to above with the result that it has now been found that dendritic polymers possess lower solution and melt viscosities due to the more globular architecture of such molecules. Dendrimers in particular have been shown to give low solution and melt viscosities and due to the perfect nature of their structures typically do not show a M_{c}. Indeed in many cases dendrimers show a decrease in solution or melt viscosity above a particular molecular weight. The synthesis of dendritic materials however is extremely tedious typically requiring a multi-step synthetic route where the ultimate molecular weights or chemical functionalities are limited. For these reasons dendrimers are extremely expensive to prepare when compared to commercially available polymers and are therefore only suitable for a limited number of high end applications.

### Branched Polymers

Branched polymers are polymer molecules of a finite size which are branched. Branched polymers differ from cross-linked polymer networks which tend towards an infinite size having interconnected molecules and which are generally not soluble. In some instances, branched polymers have advantageous properties when compared to analogous linear polymers. For example, higher molecular weights of branched copolymers can be solubilised more easily than those of corresponding linear polymers. In addition, as branched polymers tend to have more end groups than linear polymers they generally exhibit strong surface-modification properties. Branched polymers are therefore useful components of many compositions and their utilisation in a variety of applications is desirable.

The inventors have now found that branched polymers also possess lower solution or melt viscosities presumably due to their non-linear architecture. In addition, unlike dendrimers, such compounds typically exhibit non-ideal branching in structure and can possess polydisperse structures and molecular weights. Furthermore, the preparation of branched polymers is much more readily achieved than their dendrimer counterparts and although the ultimate structure of such polymers is neither perfect nor mono-disperse, branched (or hyperbranched) polymers are far more suitable and economical to produce for a variety of industrial applications.

Branched polymers are usually prepared via a step-growth mechanism via the polycondensation of suitable monomers and are usually limited by the choice of monomers, chemical functionality of the resulting polymer and the molecular weight. In addition polymerisation, a one-step process may be employed in which a multifunctional monomer is used to provide functionality in the polymer chain from which polymer branches may grow. However, a limitation on the use of a conventional one-step process is that the amount of multifunctional monomer must be carefully controlled, usually to substantially less than 0.5% w/w in order to avoid extensive cross-linking of the polymer and the formation of insoluble gels. It is difficult to avoid cross-linking using this method, especially in the absence of a solvent as a diluent and/or at high conversion of monomer to polymer.

WO 99/46301 discloses a method of preparing a branched polymer comprising the steps of mixing together a monofunctional vinylic monomer with from 0.3 to 100% w/w (of the weight of the monofunctional monomer) of a multifunctional vinylic monomer and from 0.0001 to 50% w/w (of the weight of the monofunctional monomer) of a chain transfer agent and optionally a free-radical polymerisation initiator and thereafter reacting said mixture to form a copolymer. The examples of WO 99/46301 describe the preparation of primarily hydrophobic polymers and, in particular, polymers wherein methyl methacrylate constitutes the monofunctional monomer. These polymers are useful as components in reducing the melt viscosity of linear poly(methyl methacrylate) in the production of moulding resins.

WO 99/46310 discloses a method of preparing a (meth)acrylate functionalised polymer comprising the steps of mixing together a monofunctional vinylic monomer with from 0.3 to 100% w/w (based on monofunctional monomer) of a polyfunctional vinylic monomer and from 0.0001 to 50% w/w of a chain transfer agent, reacting said mixture to form a polymer and terminating the polymerisation reaction before 99% conversion. The resulting polymers are useful as components of surface coatings and inks, as moulding resins or in curable compounds, for example curable moulding resins or photoresists.

WO 02/34793 discloses a rheology modifying copolymer composition containing a branched copolymer of an unsaturated carboxylic acid, a hydrophobic monomer, a hydrophobic chain transfer agent, a cross-linking agent, and, optionally, a steric stabilizer. The copolymer provides increased viscosity in aqueous electrolyte-containing environments at elevated pH. The method for production is a solution polymerisation process. The polymer is lightly cross-linked, less than 0.25%.

U.S. Pat. No. 6,020,291 discloses aqueous metal working fluids used as lubricants in metal cutting operations. The fluids contain a mist-suppressing branched copolymer, including hydrophobic and hydrophilic monomers, and optionally a monomer comprising two or more ethylenically unsaturated bonds. Optionally, the metal working fluid may be an oil-in-water emulsion. The polymers are based on poly(acrylamides) containing sulfonate-containing and hydrophobically modified monomers. The polymers are cross-linked to a very small extent by using very low amount of bis-acrylamide, without using a chain transfer agent.

Kim and Webster (Macromolecules, 1992, 25, 5561) describe the synthesis of polyphenylenes prepared via a step-growth AB₂ mechanism. The branched polymers were blended with linear polystyrene and the melt viscosities of the blends were found to be lower than that of the purely linear material. Some mixing issues occurred with blending, presumably due to incompatibility between polystyrene and the branched polyphenylenes, although a reduction in melt viscosity of up to 80% was measured for a 5% branched/linear polymer blend at 120° C.

Volt et al (Macromolecules 1999, 32, 6333) describe the synthesis of branched polyesters based on 3,5-dihydroxybenzoic acid prepared via a step-growth AB₂ mechanism and end-functionalised with dodecanoyl chloride to achieve an alkyl-functional material. The branched polymers were blended with linear isotactic polypropylene and high density polyethylene. Blending of the branched species with the linear polymers resulted in a reduction of the complex melt viscosity when compared to the linear polymers, this was particularly true for the polyethylene material due to the stronger interaction and higher similarity between the alkyl-modified branched polymer and the polyolefin.

WO 96/17041 describes the use of a star-branched hydrogenated polyisoprene from the “SHELLVIS” range to reduce the viscosity of a crystalline and amorphous olefin copolymer-containing lubricating oil. The branched polymer additive reduced the cold flowing of the lubricant formulation while imparting shear stability over the neat olefin copolymer formulations. The star-branched polymers are prepared via a two-step anionic polymerisation of a conjugated diene followed by reacting the living polymer core with a polyalkenyl coupling agent to form a star-shaped polymer.

WO 98/51731 describes the use of a comb-like addition polymer containing hydrophobic side chains of at least 10 carbon atoms in length in combination with a thixotropic, partly hydrated, polysaccharide for use as a viscosity reducer (pour point depressor) in wax-containing fuel oils. Although the addition polymer described is not branched, the comb-like structure induces a degree of viscosity reduction, albeit smaller than that obtained by the use of a branched polymer additive.

WO 96/23012 describes the synthesis and use of a star-branched (meth)acrylate-containing copolymer for use as a viscosity improver (reducer) for lubricating oils. The star-branched polymer is prepared via a two-step anionic polymerisation procedure where the arms or core are prepared, via the polymerisation of a mono alkenyl or polyalkenyl monomer respectively, following combination of the two species to give a star-branched polymer.

EP 0818525A2 describes the synthesis and use of a star-branched polyolefin for use as a fuel additive where the material acts as a viscosity index improver in combination with a linear ethylene-propylene copolymer. The star-branched polymers are prepared via a number of techniques including pre-forming the core and arm structures and subsequent linking or from post-modification of grafting from a branched, or dendritic pre-polymer. The star-branched polymer additive reduces the viscosity of the linear olefin-containing formulation.

WO 2008/071661 (Unilever) describes the synthesis of amphiphilic branched addition polymers and their use as emulsion stabilisers through the formation of polymer nanoparticles in solution. The materials can form amphiphilic particles via for example a pH trigger.

WO 2008/071662 (Unilever) describes the preparation of branched addition polymers wherein a component of the polymer, monofunctional monomer, polyfunctional monomer or chain transfer agent, has a molecular weight of at least 1000 Daltons. The polymers are described as aiding the colloid stabilisation in laundry formulations.

### DETAILED DESCRIPTION

Polymers are ubiquitous in their everyday usage. A common application of these materials is as viscosity modifiers in solution where they essentially thicken many formulations ranging from shower gels to topical pharmaceutical products. In these applications the intrinsic high molecular weight of the polymers, molecular association, chain entanglement and ultimate rise in the formulation viscosity is advantageous. In many applications however, a low viscosity formulation is desirable and many different routes have been used to achieve this including: increasing the amount of solvent, the addition of a low molecular weight viscosity reducer, the use of a “reactive diluent” or by heating the formulation. In applications such as in coatings, lubricants or adhesives the reduction in solvent while maintaining an equivalent solution viscosity is particularly attractive. Here the polymer usually imparts key benefits such as film-formation, curing and adhesion or friction reduction. The reduction in volatile organic compounds (VOCs) is however a key driver in many industrial applications, driven by environmental legislation or cost savings. By reducing the volatile organic compound (VOC) content and thereby preparing solutions of greater solids content, that is, concentrates, significant savings may also be made during the transportation of formulations containing the polymeric materials. Where the polymer is used in a solution formulation, improved solubility is also an advantage.

The addition of low molecular weight polymers has been tried as a means of reducing the solution or melt viscosity of polymer formulations. Here the oligomeric species essentially plasticises the formulation or reduces the intermolecular forces which lead to viscosity increases in the bulk polymer, such as chain entanglement or H-bonding. The use of these small molecular weight additives can be problematic however as it can be difficult to achieve miscibility with the bulk polymer if the additive is chemically distinct; such as when blending a low molecular weight polyester with a high molecular weight addition polymer such as polystyrene. Where the ultimate end-use requires good film properties, such as in coatings, this can lead to the so called “orange peel” effect. Additionally the final properties of the application can be affected by the use of a small molecular weight additive, resulting in poor performance.

It has also been found that the addition of dendritic polymers to a linear polymer-containing formulation can lead to a reduction in melt or solution viscosity of the formulation. In many cases small amounts of these additives is required leading to minimal effect on the performance and overall composition of the formulation. Additionally, due to the dendritic or highly branched architectures of such additives, they can possess equivalent molecular weights to the bulk linear polymer leading to little or no reduction in the average molecular weight of the polymers.

However, dendritic polymers are prepared via a multi-step synthetic route and are limited by chemical functionality and ultimate molecular weight, being prepared at a high cost. Dendritic polymers have therefore only limited high-end industrial applications.

Whilst branched polymers are typically prepared via a step-growth procedure and are sometimes limited by their chemical functionality and molecular weight, their reduced cost makes them more industrially attractive. In addition, the chemical nature of dendritic polymers means that such polymers typically possess ester or amide linkages, with the result that some issues regarding the miscibility of these polymers with olefin-derived polymers have been observed. Whilst this can be circumvented by the use of hydrocarbon-based, star-shaped polymers prepared via anionic polymerisation or the post-functionalisation of pre-formed dendrimers species this leads to an increased cost in the materials.

There is therefore a need for high molecular weight polymers possessing low solution or melt viscosities at a viable cost.

Additionally a reduction in the melt or solution elasticity behaviour of a blend of linear and branched addition polymer compared to a linear polymer provides a particular advantage in terms of the ease of application of the formulation via jetting, injection, spraying or roller applying. Additionally, the reduced elastic behaviour of the blend formulation can lead to benefits in pumping or processing melt or solutions of branched addition polymers.

Through previous disclosures the inventors have shown that branched copolymers of high molecular weight can be prepared via a one-step process using commercially available monomers such as in WO 2008/071662 the contents of which are incorporated herein by reference. Through specific monomer choices the inventors have found that the chemical functionality of these polymers can be tuned depending on the specific application area. These findings therefore give advantages over dendritic or step-growth branched polymers. Since the branched addition copolymers are prepared via an addition process from commercially available monomers, they can be ‘tuned’ to give good miscibility with equivalent linear addition polymers. Also, since the branched addition polymers comprise a carbon-carbon backbone they are not susceptible to thermal or hydrolytic instability unlike ester-based dendrimers or step-growth hyperbranched polymers. It has further been observed that these polymers also dissolve faster than equivalent linear polymers.

The branched copolymers of the present invention are branched, non-cross-linked addition polymers and include statistical, block, graft, gradient and alternating branched copolymers. The copolymers of the present invention comprise at least two chains which are covalently linked by a bridge other than at their ends, that is, a sample of said copolymer comprises on average at least two chains which are covalently linked by a bridge other than at their ends. When a sample of the copolymer is made there may be unintentionally some polymer molecules that are un-branched, which is inherent to the production method (addition polymerisation process). For the same reason, a small quantity of the polymer will not have a chain transfer agent (CTA) on the chain end.

There is therefore a need for a form of polymer which can be readily produced at a reasonable cost on an industrial scale and which is able to be included in a polymeric formulation thereby replacing a quantity of a linear polymer, and hence leading to a reduction in solution or melt viscosity when compared to a formulation containing only linear polymeric material.

Also surprisingly, the inventors have now found that the use of branched addition copolymers in this way may achieve a reduction in solution or melt viscosity of a polymeric formulation when the viscosity of the formulation is compared to a polymeric formulation comprising only linear polymeric material. It has also been found that the use of branched addition copolymers as viscosity reducing additives produces formulations of reduced solution or melt viscosity and with a higher solids content than a formulation consisting solely linear polymeric material.

Also, and more importantly, there is a requirement for a form of polymer which can be readily produced at a reasonable cost on an industrial scale and which is able to be included into a formulation which ultimately reduces the solution or melt viscosity of a formulation and/or which can lead to a formulation of increased solids content.

Therefore according to a first aspect of the present invention there is provided the use of a branched addition copolymer in combination with a polymer in a solution or melt formulation to reduce the viscosity of the solution formulation and/or melt formulation compared to the viscosity of a solution and/or melt comprising the polymer alone wherein the branched addition copolymer is obtainable by an addition polymerisation process.

In addition in relation to the first aspect of the present invention provides the use of a branched addition copolymer in a solution formulation or melt formulation to form a blend in combination with an analogous linear copolymer to reduce the viscosity of the solution and/or melt formulation compared to the viscosity of a solution and/or melt comprising an equivalent analogous linear polymer with comparable weight average molecular weight alone wherein the branched addition copolymer is obtainable by an addition polymerisation process.

In addition the use of a branched polymer according to the first aspect of the present invention in a solution formulation or melt formulation is able to form a blend in combination with an analogous linear copolymer to reduce the viscosity of the solution formulation and/or melt formulation compared to the viscosity of a solution formulation and/or melt comprising an equivalent analogous linear polymer, wherein the weight average molecular weight of the blend is at least 5% higher than the solution formulation or melt formulation of the linear polymer alone.

Also according to the first aspect of the present invention the branched addition polymer preferably comprises between and 1 and 99% of the blend. More preferably the branched addition polymer comprises between and 1 and 70% of the blend. Even more preferably the branched addition polymer comprises between and 1 and 50% of the blend.

Also in relation to the first aspect of the present invention the branched addition polymer has a weight average molecular weight of 2,000 Da to 1,500,000 Da. More preferably the branched addition polymer comprises a weight average molecular weight of 5,000 Da to 1,000,000 Da. Most preferably the branched addition polymer comprises a weight average molecular weight of 5,000 Da to 700,000 Da.

The branched addition copolymer for use in accordance with a first aspect of the present invention comprises:
at least two chains which are covalently linked by a bridge other than at their ends; and wherein
at least two chains comprise at least one ethyleneically monounsaturated monomer, and wherein
the bridge comprises at least one ethyleneically polyunsaturated monomer; and wherein
the polymer comprises a residue of a chain transfer agent and/or optionally a residue of an initiator; and wherein
the mole ratio of polyunsaturated monomer(s) to monounsaturated monomer(s) is in a range of from 1:100 to 1:4.

More preferably the branched addition copolymer for use in accordance with a first aspect of the present invention comprises:
at least two chains which are covalently linked by a bridge other than at their ends; and wherein
at least two chains comprise at least one ethyleneically monounsaturated monomer, and wherein
the bridge comprises at least one ethyleneically polyunsaturated monomer; and wherein
the polymer comprises a residue of a chain transfer agent and/or optionally a residue of an initiator; and wherein
at least one of the monounsaturated monomer(s) and polyunsaturated monomer(s) and chain transfer agent(s) is a hydrophilic residue; and
at least one of one of the monounsaturated monomer(s) and polyunsaturated monomer(s) and chain transfer agent(s) is a hydrophobic residue; and wherein the mole ratio of polyunsaturated monomer(s) to monounsaturated monomer(s) is in a range of from 1:100 to 1:4.

It is also preferred that in relation to the first aspect of the present invention the branched addition copolymer comprises less than 1% monomer impurity.

Furthermore, by using the branched addition copolymer according to the first aspect of the present invention the branched addition copolymer is able to provide solutions of at least 5% higher solids content with equivalent solution viscosity than a linear polymer equivalent.

Also, when the blend is processed or melted the branched addition copolymer used in accordance with the first aspect of the present invention is able to further provide a reduction in the melt temperature or processing temperature of at least 5% compared to the polymer equivalent.

It has also been found that the use of the branched addition copolymers in relation to the first aspect of the present invention may be utilised to reduce the viscosity of a solution and/or melt such that wherein the branched addition copolymer is added to a solution and/or melt comprising a linear polymer according to Equation 1

ηBlend=η_{Bp}^{α}η_{LP}^{(1-α)} Equation 1

wherein: in Equation 1 relates to a theoretical relationship of a blend of two polymers, in this case a branched copolymer and a linear polymer of varying solution viscosities and
α—is the weight fraction of the branched copolymer and
η_{BP} is the viscosity of the branched copolymer solution at the same solids content; and
η_{LP} is the viscosity of the linear polymer solution at the same solids content and the value of ηBlend is the measured viscosity of the blend.

The use of the branched addition copolymers according to the first aspect of the present invention find specific application in reducing the viscosity of a solution and/or melt in the application areas selected from the group comprising:
coatings, inks, adhesives, lubricants, composites, oil field recovery agents, metal working fluids, coolants, sealants, films, resins, textiles, injection mouldings, water treatment, electronics, cosmetics, pharmaceuticals, agrochemicals and lithography.

According to a second aspect of the present invention there is provided a polymer blend comprising a branched addition copolymer described in relation to the first aspect of the present invention wherein the copolymer is obtainable by an addition polymerisation process, and wherein the branched copolymer comprises a weight average molecular weight of 2,000 Da to 1,500,000 Da and wherein the branched addition copolymer comprises:
at least two chains which are covalently linked by a bridge other than at their ends; and wherein
at least two chains comprise at least one ethyleneically monounsaturated monomer, and wherein
the bridge comprises at least one ethyleneically polyunsaturated monomer; and wherein
the polymer comprises a residue of a chain transfer agent and/or optionally a residue of an initiator; and wherein
at least one of the monounsaturated monomer(s) and polyunsaturated monomer(s) and chain transfer agent(s) is a hydrophilic residue; and
at least one of one of the monounsaturated monomer(s) and polyunsaturated monomer(s) and chain transfer agent(s) is a hydrophobic residue; and wherein
the mole ratio of polyunsaturated monomer(s) to monounsaturated monomer(s) is in a range of from 1:100 to 1:4, and an linear equivalent polymer wherein the blend comprises between 10 and 90% branched addition copolymer and between 90 and 10% linear equivalent polymer.

The monomers used in the polymer blend to prepare the branched addition copolymers in the second aspect of the present invention are vinylic or allylic in nature and are selected from the group comprising: styrenics, acrylics, methacrylics, allylics, acrylamides, methacrylamides, vinyl or allyl acetates, N-vinyl or allyl amines and vinyl or allyl ethers.

Preferred branched addition copolymers according to the first and second aspects of the present invention contain units selected from the groups consisting of: styrene, vinyl benzyl chloride, 2-vinyl pyridine, 4-vinyl pyridine, methyl acrylate, ethyl acrylate, methyl methacrylate, ethyl methacrylate, butyl methacrylate, butyl acrylate, acrylic acid, methacrylic acid, 2-hydroxyethyl methacrylate, 2-hydroxyethyl acrylate, 2-hydroxypropyl acrylate, 2-hydroxypropyl methacrylate, acrylamide, methacrylamide, dimethyl acrylamide, dimethyl(meth)acrylamide, allyl methacrylate, dimethylaminoethyl methacrylate, dimethylaminoethyl acrylate, diethylaminoethyl methacrylate, diethylaminoethyl acrylate, divinyl benzene, ethyleneglycol dimethacrylate, ethyleneglycol diacrylate, triethylene glycol dimethacrylate, tetraethyleneglycol dimethacrylate, triethyleneglycol diacrylate, tetraethyleneglycol diacrylate, 1,3,5-triallyl-1,3,5-triazine-2,4,6(1H,3H,5H)-trione, dodecane thiol, hexane thiol, 2-mercaptoethanol and fragments arising from azobis isobutyronitrile, di-t-butyl peroxide and t-butyl peroxybenzoate.

More preferably the branched addition copolymers according to the first and second aspects of the present invention contain units selected from the groups consisting of: styrene, 2-vinyl pyridine, 4-vinyl pyridine, methyl acrylate, methyl methacrylate, butyl methacrylate, butyl acrylate, acrylic acid, methacrylic acid, acrylamide, methacrylamide, dimethyl acrylamide, dimethyl(meth)acrylamide, divinyl benzene, ethyleneglycol dimethacrylate, ethyleneglycol diacrylate, triethylene glycol dimethacrylate, dodecane thiol, hexane thiol, 2-mercaptoethanol, azobis iso butyronitrile, di-t-butyl peroxide and t-butyl peroxybenzoate.

Also, in accordance with a third aspect of the present invention there is provided a branched addition copolymer as described in relation to the second aspect of the present invention and a linear equivalent polymer as described in relation to the first aspect of the present invention and a liquid medium wherein the liquid medium comprises an organic solvent and/or an aqueous solvent. The formulation is used to reduce the solution viscosity of a solution comprising an equivalent linear polymer according to a first aspect of the present invention. The branched copolymer can also be used as a direct replacement for a linear analogue in a melt formulation with or without the use of a solvent.

Finally in relation to the third aspect of the present invention the formulation may be used to reduce the solution and/or melt viscosity of a solution and/or melt comprising an equivalent linear polymer by 20%.

It has been found that in accordance with the first aspect of the present invention the branched addition copolymers may be used for a variety of applications including but not limited to:

i) The use of the branched addition copolymers in formulations for coatings, where the low viscosity of the branched addition copolymer used as a replacement additive can lead to high solids content formulations with lower volatile organic compounds (VOCs) content than the linear equivalent polymers, at the same solids content. Additional advantages include faster drying times and ease of application of the polymer. That is higher solids content or lower viscosity coatings can be formulated through the incorporation of the described branched addition copolymers resulting in a formulation which can provide faster drying, easier application and with a reduction in the VOC content of the formulation.
ii) The use of the branched addition copolymers in for example ink formulations. This again leads to the production of lower viscosity inks, with the resultant ability to incorporate a higher ‘payload’ of pigments, dyes or other adjuncts into the ink formulations.
iii) Use of the branched addition copolymers in adhesives, wherein the use of said branched addition copolymers leads to lower viscosity adhesives with a higher composition of curable or pressure-sensitive adhesive actives present in the adhesive composition.
iv) Use of the branched addition copolymers as lubricants, wherein a higher proportion of lubricating branched addition copolymers in a formulation leads to greater friction reducing power with improved viscosity indices. For example, incorporating branched addition copolymers into a lubricant formulation whereby the formulation can be processed with higher solids content and can result in increased friction reduction at high shear and higher temperatures.
v) Use of the branched addition copolymers as composites, wherein the use of a low viscosity branched copolymer in a composite is able to improve the penetration of the polymer into the composite matrix aiding cure levels and increasing the final strength of the polymer.
vi) Use of the branched addition copolymers as oil field recovery agents, where it is a) required to prepare formulations with a higher solids content and b) less deformable recovery agents for oil-field applications with improved, that is, more Newtonian viscosity profiles.
vii) Use of the branched addition copolymers in cutting fluids, wherein the branched addition copolymers provide a means to achieve higher solids content in the cutting fluids with improved friction reduction and heat transfer capabilities.
viii) Use of the branched addition copolymers in coolants, where the use of the branched addition copolymers provides coolant formulations with lower viscosities providing easier transport and pumping characteristics.
ix) Use of the branched addition copolymers in sealant formulations wherein high modulus sealants are prepared and wherein the formulations have the advantages of higher cure rate, lower volatile organic compound (VOC) levels and greater overall strength due to the high solids content. In addition, the branched addition copolymers may be used to produce low viscosity sealant foam formulations containing low levels of volatile organic compounds (VOCs).
x) Use of the branched addition copolymers in the production of films wherein low volatile organic compound (VOC) levels and high solids content films may be cast using branched copolymers wherein the production of fast film formation is an advantage. In addition melt processing to blow films can be achieved at lower process temperatures.
xi) Use of the branched addition copolymers in resins, wherein efficient solution or melt processing of resins can be achieved as a result of the use of said copolymers. In solution processing the key advantages are the preparation of high solids formulations with low viscosities and low volatile organic compounds (VOCs). In melt processing, lower production temperatures can also be achieved as a result of the presence of the polymers.
xii) The branched addition copolymers of the present invention may also be used for textile applications where low viscosity, low volatile organic compound levels (VOC), with high solids content for textile treatments comprising the branched addition copolymers is required. This leads to greater textile penetration of the polymer. In melt-spinning techniques, lower temperatures are also achievable by using the branched addition copolymers in the textile formulations.
xiii) The branched addition copolymers may also be used in injection moulding techniques where the use of a branched addition copolymer leads to a reduction in the process times and temperatures required due to the lower melt viscosity.
xiv) The branched addition copolymers may be used in lithography applications wherein the use of the branched addition copolymers with reduced viscosity can be utilised as a resist in lithography, whereby the lower viscosity aids the formation of more precise templates or structures.
xv) The branched addition copolymer described in accordance with the present invention may also be used in place of a linear equivalent polymer in a solution or melt to achieve a solution or melt with reduced elastic behaviour. The branched copolymer materials may then be used at higher solids level in for example a solution formulation, whereby the formulation has a lower elastic profile. As a result such formulations may be sprayed, rolled, injected, mixed, and processed much easier and with less energy input than equivalent linear polymeric materials. This is also true when the polymeric materials are in melt form with the consequence that much lower melt temperatures may be used.
xvi) Finally, the branched addition copolymers may also be used in applications where the lower elastic behaviour of the branched polymer formulation results in generally an easier application of a solution or melt of the branched polymer such as by extrusion, injection, jet-application or spraying compared to linear polymers. This can result in faster application times and lower temperatures required for melt applications of higher solids content of the polymer in a solution formulation.

It will be appreciated that in all of the uses described above the branched addition copolymers are used as an additive in the total polymeric formulation, that is, the branched addition copolymers are used in a blend of linear and branched copolymer.

The chain transfer agent (CTA) is a molecule which is known to reduce molecular weight during a free-radical polymerisation via a chain transfer mechanism. These agents may be any thiol-containing molecule and can be either monofunctional or multifunctional. The agent may be hydrophilic, hydrophobic, amphiphilic, anionic, cationic, neutral, zwitterionic or responsive. The molecule can also be an oligomer or a pre-formed polymer containing a thiol moiety. (The agent may also be a hindered alcohol or similar free-radical stabiliser). Catalytic chain transfer agents such as those based on transition metal complexes such as cobalt bis(borondifluorodimethyl-glyoximate) (CoBF) may also be used. Suitable thiols include but are not limited to: C₂ to C₁₈ branched or linear alkyl thiols such as dodecane thiol, functional thiol compounds such as thioglycolic acid, thio propionic acid, thioglycerol, cysteine and cysteamine. Thiol-containing oligomers or polymers may also be used such as for example poly(cysteine) or an oligomer or polymer which has been post-functionalised to give a thiol group(s), such as poly(ethyleneglycol) (di)thio glycollate, or a pre-formed polymer functionalised with a thiol group. For example, the reaction of an end or side-functionalised alcohol such as poly(propylene glycol) with thiobutyrolactone, will give the corresponding thiol-functionalised chain-extended polymer. Multifunctional thiols may also be prepared by the reduction of a xanthate, dithioester or trithiocarbonate end-functionalised polymer prepared via a Reversible Addition Fragmentation Transfer (RAFT) or Macromolecular Design by the Interchange of Xanthates (MADIX) living radical method. Xanthates, dithioesters, and dithiocarbonates may also be used, such as cumyl phenyldithioacetate. Alternative chain transfer agents may be any species known to limit the molecular weight in a free-radical addition polymerisation including alkyl halides, ally-functional compounds and transition metal salts or complexes. More than one chain transfer agent may be used in combination.

Hydrophobic CTAs include but are not limited to linear and branched alkyl and aryl (di)thiols such as dodecanethiol, octadecyl mercaptan, 2-methyl-1-butanethiol and 1,9-nonanedithiol. Hydrophobic macro-CTAs (where the molecular weight of the CTA is at least 1000 Daltons) can be prepared from hydrophobic polymers synthesised by RAFT (or MADIX) followed by reduction of the chain end, or alternatively the terminal hydroxyl group of a preformed hydrophobic polymer can be post functionalised with a compound such as thiobutyrolactone. Non-thiol CIA's such as 2,4-diphenyl-4-methyl-1-pentene can also be used.

Hydrophilic CTAs typically contain hydrogen bonding and/or permanent or transient charges. Hydrophilic CTAs include but are not limited to: thio-acids such as thioglycolic acid and cysteine, thioamines such as cysteamine and thio-alcohols such as 2-mercaptoethanol, thioglycerol and ethylene glycol mono- (and di-)thio glycollate. Hydrophilic macro-CTAs (where the molecular weight of the CTA is at least 1000 Daltons) can also be prepared from hydrophilic polymers synthesised by RAFT (or MADIX) followed by reduction of the chain end, or alternatively the terminal hydroxyl group of a preformed hydrophilic polymer can be post functionalised with a compound such as thiobutyrolactone.

Amphiphilic CTAs can also be incorporated in the polymerisation mixture, these materials are typically hydrophobic alkyl-containing thiols possessing a hydrophilic function such as but not limited to a carboxylic acid group. Molecules of this type include mercapto undecylenic acid.

Responsive macro-CTAs (where the molecular weight of the CTA is at least 1000 Daltons) can be prepared from responsive polymers synthesised by RAFT (or MADIX) followed by reduction of the chain end, or alternatively the terminal hydroxyl group of a preformed responsive polymer, such as poly(propylene glycol), can be post functionalised with a compound such as thiobutyrolactone.

The residue of the chain transfer agent may comprise 0 to 80 mole % of the copolymer (based on the number of moles of monofunctional monomer). More preferably the residue of the chain transfer agent comprises 0 to 50 mole %, even more preferably 0 to 40 mole % of the copolymer (based on the number of moles of monofunctional monomer). However, most especially the chain transfer agent comprises 0.05 to 30 mole %, of the copolymer (based on the number of moles of monofunctional monomer).

The initiator is a free-radical initiator and can be any molecule known to initiate free-radical polymerisation such as for example azo-containing molecules, persulfates, redox initiators, peroxides, benzyl ketones. These may be activated via thermal, photolytic or chemical means. Examples of these include but are not limited to: 2,2′-azobisisobutyronitrile (AIBN), azobis(4-cyanovaleric acid), benzoyl peroxide, diisopropyl peroxide, t-butyl peroxybenzoate (for example Luperox® P), di-t-butyl peroxide (for example Luperox® DI), cumylperoxide, 1-hydroxycyclohexyl phenyl ketone, hydrogenperoxide/ascorbic acid. Iniferters such as benzyl-N,N-diethyldithiocarbamate can also be used. In some cases, more than one initiator may be used. The initiator may be a macroinitiator having a molecular weight of at least 1000 Daltons. In this case, the macroinitiator may be hydrophilic, hydrophobic, or responsive in nature.

Preferably, the residue of the initiator in a free-radical polymerisation comprises from 0 to 10% w/w of the copolymer based on the total weight of the monomers. More preferably the residue of the initiator in a free-radical polymerisation comprises from 0.001 to 8% w/w of the copolymer, and especially 0.001 to 5% w/w, of the copolymer based on the total weight of the monomers.

The use of a chain transfer agent and an initiator is preferred. However, some molecules can perform both functions.

Hydrophilic macroinitiators (where the molecular weight of the pre-formed polymer is at least 1000 Daltons) can be prepared from hydrophilic polymers synthesised by RAFT (or MADIX), or a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

Hydrophobic macroinitiators (where the molecular weight of the preformed polymer is at least 1000 Daltons) can be prepared from hydrophobic polymers synthesised by RAFT (or MADIX), or a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

Responsive macroinitiators (where the molecular weight of the preformed polymer is at least 1000 Daltons) can be prepared from responsive polymers synthesised by RAFT (or MADIX), or a functional group of a preformed hydrophilic polymer, such as terminal hydroxyl group, can be post-functionalised with a functional halide compound, such as 2-bromoisobutyryl bromide, for use in Atom Transfer Radical Polymerisation (ATRP) with a suitable low valency transition metal catalyst, such as CuBr Bipyridyl.

The monofunctional monomer may comprise any carbon-carbon unsaturated compound which can be polymerised by an addition polymerisation mechanism, for example vinyl and allyl compounds. The monofunctional monomer may be hydrophilic, hydrophobic, amphiphilic, anionic, cationic, neutral or zwitterionic in nature. The monofunctional monomer may be selected from but not limited to monomers such as:

vinyl acids, vinyl acid esters, vinyl aryl compounds, vinyl acid anhydrides, vinyl amides, vinyl ethers, vinyl amines, vinyl aryl amines, vinyl nitriles, vinyl ketones, and derivatives of the aforementioned compounds as well as corresponding allyl variants thereof.

Other suitable monofunctional monomers include: hydroxyl-containing monomers and monomers which can be post-reacted to form hydroxyl groups, acid-containing or acid-functional monomers, zwitterionic monomers and quaternised amino monomers. Oligomeric, polymeric and di- or multi-functionalised monomers may also be used, especially oligomeric or polymeric (meth)acrylic acid esters such as mono(alkyl/aryl) (meth)acrylic acid esters of polyalkyleneglycol or polydimethylsiloxane or any other mono-vinyl or allyl adduct of a low molecular weight oligomer. Mixtures of more than one monomer may also be used to give statistical, graft, gradient or alternating copolymers.

Vinyl acids and derivatives thereof include: (meth)acrylic acid, fumaric acid, maleic acid, vinyl sulfonic acid, vinyl phosphoric acid, 2-acrylamido-2-methylpropane sulfonic acid, itaconic acid and acid halides thereof such as (meth)acryloyl chloride. Vinyl acid esters and derivatives thereof include: C₁ to C₂₀ alkyl(meth)acrylates (linear and branched) such as for example methyl(meth)acrylate, stearyl (meth)acrylate and 2-ethyl hexyl(meth)acrylate; aryl(meth)acrylates such as for example benzyl(meth)acrylate; tri(alkyloxy)silylalkyl(meth)acrylates such as trimethoxysilylpropyl(meth)acrylate; and activated esters of (meth)acrylic acid such as N-hydroxysuccinamido(meth)acrylate.

Vinyl aryl compounds and derivatives thereof include: styrene, acetoxystyrene, styrene sulfonic acid, 2- and 4-vinyl pyridine, vinyl naphthalene, vinylbenzyl chloride and vinyl benzoic acid.

Vinyl acid anhydrides and derivatives thereof include: maleic anhydride. Vinyl amides and derivatives thereof include: (meth)acrylamide, N-(2-hydroxypropyl)methacrylamide, N-vinyl pyrrolidone, N-vinyl formamide, (meth)acrylamidopropyl trimethyl ammonium chloride, [3-((meth)acrylamido)propyl]dimethyl ammonium chloride, 3-[N-(3-(meth)acrylamidopropyl)-N,N-dimethyl]aminopropane sulfonate, methyl (meth)acrylamidoglycolate methyl ether and N-isopropyl(meth)acrylamide.

Vinyl ethers and derivatives thereof include: methyl vinyl ether.

Vinyl amines and derivatives thereof include: dimethylaminoethyl(meth)acrylate, diethylaminoethyl(meth)acrylate, diisopropylaminoethyl(meth)acrylate, mono-t-butylaminoethyl (meth)acrylate, morpholinoethyl(meth)acrylate and monomers which can be post-reacted to form amine groups, such as N-vinyl formamide.

Vinyl aryl amines and derivatives thereof include: vinyl aniline, 2 and 4-vinyl pyridine, N-vinyl carbazole and vinyl imidazole.

Vinyl nitriles and derivatives thereof include: (meth)acrylonitrile.

Vinyl ketones or aldehydes and derivatives thereof include: acrolein.

Hydroxyl-containing monomers include:

vinyl hydroxyl monomers such as hydroxyethyl(meth)acrylate, 1- and 2-hydroxy propyl(meth)acrylate, glycerol mono(meth)acrylate and sugar mono(meth)acrylates such as glucose mono(meth)acrylate,
monomers which can be post-reacted to form hydroxyl groups include: vinyl acetate, acetoxystyrene and glycidyl(meth)acrylate,
acid-containing or acid functional monomers include: (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid, mono-2-((meth)acryloyloxy)ethyl succinate and ammonium sulfatoethyl(meth)acrylate.

Zwitterionic monomers include: (meth)acryloyl oxyethylphosphoryl choline and betaines, such as [2-((meth)acryloyloxy)ethyl]dimethyl-(3-sulfopropyl)ammonium hydroxide.

Quaternised amino monomers include: (meth)acryloyloxyethyltri-(alkyl/aryl)ammonium halides such as (meth)acryloyloxyethyltrimethyl ammonium chloride.

Vinyl acetate and derivatives thereof can also be utilised.

Oligomeric and polymeric monomers include: oligomeric and polymeric (meth)acrylic acid esters such as mono(alkyl/aryl)oxypolyalkyleneglycol(meth)acrylates and mono(alkyl/aryl)oxypolydimethyl-siloxane(meth)acrylates. These esters include for example: monomethoxy oligo(ethyleneglycol)mono(meth)acrylate, monomethoxy oligo(propyleneglycol)mono(meth)acrylate, monohydroxy oligo(ethyleneglycol) mono(meth)acrylate, monohydroxy oligo(propyleneglycol)mono(meth)acrylate, monomethoxy poly(ethyleneglycol)mono(meth)acrylate, monomethoxy poly(propyleneglycol)mono(meth)acrylate, monohydroxy poly(ethyleneglycol) mono(meth)acrylate and monohydroxy poly(propyleneglycol)mono(meth)acrylate.

Further examples include: vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as poly(1,4-butadiene).

The corresponding allyl monomers to those listed above can also be used where appropriate.

Examples of monofunctional monomers are:

amide-containing monomers such as (meth)acrylamide, N-(2-hydroxypropyl)methacrylamide, N,N′-dimethyl(meth)acrylamide, N and/or N′-di(alkyl or aryl)(meth)acrylamide, N-vinyl pyrrolidone, [3-((meth)acrylamido)propyl]trimethyl ammonium chloride, 3-(dimethylamino)propyl(meth)acrylamide, 3-[N-(3-(meth)acrylamidopropyl)-N,N-dimethyl]aminopropane sulfonate, methyl (meth)acrylamidoglycolate methyl ether and N-isopropyl(meth)acrylamide; (meth)acrylic acid and derivatives thereof such as (meth)acrylic acid, (meth)acryloyl chloride (or any halide), (alkyl/aryl)(meth)acrylate; functionalised oligomeric or polymeric monomers such as monomethoxy oligo(ethyleneglycol) mono(meth)acrylate, monomethoxy oligo(propyleneglycol)mono(meth)acrylate, monohydroxy oligo(ethyleneglycol)mono(meth)acrylate, monohydroxy oligo(propyleneglycol)mono(meth)acrylate, monomethoxy poly(ethyleneglycol) mono(meth)acrylate, monomethoxy poly(propyleneglycol)mono(meth)acrylate, monohydroxy poly(ethyleneglycol)mono(meth)acrylate, monohydroxy poly(propyleneglycol)mono(meth)acrylate, glycerol mono(meth)acrylate and sugar mono(meth)acrylates such as glucose mono(meth)acrylate;
vinyl amines such as aminoethyl(meth)acrylate, dimethylaminoethyl(meth)acrylate, diethylaminoethyl(meth)acrylate, diisopropylaminoethyl(meth)acrylate, mono-t-butylamino (meth)acrylate, morpholinoethyl(meth)acrylate; vinyl aryl amines such as vinyl aniline, vinyl pyridine, N-vinyl carbazole, vinyl imidazole, and monomers which can be post-reacted to form amine groups, such as vinyl formamide;
vinyl aryl monomers such as styrene, vinyl benzyl chloride, vinyl toluene, α-methyl styrene, styrene sulfonic acid, vinyl naphthalene and vinyl benzoic acid;
vinyl hydroxyl monomers such as hydroxyethyl(meth)acrylate, hydroxy propyl (meth)acrylate, glycerol mono(meth)acrylate or monomers which can be post-functionalised into hydroxyl groups such as vinyl acetate, acetoxy styrene and glycidyl(meth)acrylate;
acid-containing monomers such as (meth)acrylic acid, styrene sulfonic acid, vinyl phosphonic acid, vinyl benzoic acid, maleic acid, fumaric acid, itaconic acid, 2-(meth)acrylamido 2-ethyl propanesulfonic acid, vinyl sulfonic acid, vinyl phosphoric acid, 2-acrylamido-2-methylpropane sulfonic acid, and mono-2-((meth)acryloyloxy)ethyl succinate or acid anhydrides such as maleic anhydride;
zwitterionic monomers such as (meth)acryloyl oxyethylphosphoryl choline and betaine-containing monomers, such as [2-((meth)acryloyloxy)ethyl]dimethyl-(3-sulfopropyl)ammonium hydroxide;
quaternised amino monomers such as (meth)acryloyloxyethyltrimethyl ammonium chloride.
vinyl acetate or vinyl butanoate or derivatives thereof.

The corresponding allyl monomer, where applicable, can also be used in each case.

Functional monomers, that is monomers with reactive pendant groups which can be pre or post-modified with another moiety following polymerisation can also be used such as for example glycidyl(meth)acrylate, tri(alkoxy)silylalkyl(meth)acrylates such as trimethoxysilylpropyl(meth)acrylate, (meth)acryloyl chloride, maleic anhydride, hydroxyalkyl(meth)acrylates, (meth)acrylic acid, vinylbenzyl chloride, activated esters of (meth)acrylic acid such as N-hydroxysuccinamido(meth)acrylate and acetoxystyrene.

Macromonomers (monomers having a molecular weight of at least 1000 Daltons) are generally formed by linking a polymerisable moiety, such as a vinyl or allyl group, to a pre-formed monofunctional polymer via a suitable linking unit such as an ester, an amide or an ether. Examples of suitable polymers include: mono functional poly(alkylene oxides) such as monomethoxy[poly(ethyleneglycol)] or monomethoxy[poly(propyleneglycol)], silicones such as poly(dimethylsiloxane)s, polymers formed by ring-opening polymerisation such as poly(caprolactone) or poly(caprolactam) or mono-functional polymers formed via living polymerisation such as poly(1,4-butadiene).

Preferred macromonomers include: monomethoxy[poly(ethyleneglycol)]mono(methacrylate), monomethoxy[poly(propyleneglycol)]mono(methacrylate) and mono(meth)acryloxypropyl-terminated poly(dimethylsiloxane).

When the monofunctional monomer is providing the necessary hydrophilicity in the copolymer, it is preferred that the monofunctional monomer is a residue of a hydrophilic monofunctional monomer, preferably having a molecular weight of at least 1000 Daltons.

Hydrophilic monofunctional monomers include: (meth)acryloyl chloride, N-hydroxysuccinamido (meth)acrylate, styrene sulfonic acid, maleic anhydride, (meth)acrylamide, N-(2-hydroxypropyl)methacrylamide, N-vinyl pyrrolidinone, N-vinyl formamide, quaternised amino monomers such as (meth)acrylamidopropyl trimethyl ammonium chloride, [3-((meth)acrylamido)propyl]trimethyl ammonium chloride and (meth)acryloyloxyethyltrimethyl ammonium chloride, 3-[N-(3-(meth)acrylamidopropyl)-N,N-dimethyl]aminopropane sulfonate, methyl (meth)acrylamidoglycolate methyl ether, glycerol mono(meth)acrylate, monomethoxy and monohydroxy oligo(ethylene oxide) (meth)acrylate, sugar mono(meth)acrylates such as glucose mono(meth)acrylate, (meth)acrylic acid, vinyl phosphonic acid, fumaric acid, itaconic acid, 2-(meth)acrylamido 2-ethyl propanesulfonic acid, mono-2-((meth)acryloyloxy)ethyl succinate, ammonium sulfatoethyl(meth)acrylate, (meth)acryloyl oxyethylphosphoryl choline and betaine-containing monomers such as [2-((meth)acryloyloxy)ethyl]dimethyl-(3-sulfopropyl)ammonium hydroxide.

Hydrophilic macromonomers may also be used and include: monomethoxy and monohydroxy poly(ethylene oxide) (meth)acrylate and other hydrophilic polymers with terminal functional groups which can be post-functionalised with a polymerisable moiety such as (meth)acrylate, (meth)acrylamide or styrenic groups.

Hydrophobic monofunctional monomers include: C₁ to C₂₈ alkyl(meth)acrylates (linear and branched) and (meth)acrylamides, such as methyl(meth)acrylate and stearyl(meth)acrylate, aryl(meth)acrylates such as benzyl(meth)acrylate, tri(alkyloxy)silylalkyl(meth)acrylates such as trimethoxysilylpropyl(meth)acrylate, styrene, acetoxystyrene, vinylbenzyl chloride, methyl vinyl ether, vinyl formamide, (meth)acrylonitrile, acrolein, 1- and 2-hydroxy propyl(meth)acrylate, vinyl acetate, 5-vinyl 2-norbornene, Isobornyl methacrylate and glycidyl(meth)acrylate.

Hydrophobic macromonomers may also be used and include: monomethoxy and monohydroxy poly(butylene oxide) (meth)acrylate and other hydrophobic polymers with terminal functional groups which can be post-functionalised with a polymerisable moiety such as (meth)acrylate, (meth)acrylamide or styrenic groups.

Responsive monofunctional monomers include: (meth)acrylic acid, 2- and 4-vinyl pyridine, vinyl benzoic acid, N-isopropyl(meth)acrylamide, tertiary amine (meth)acrylates and (meth)acrylamides such as 2-(dimethyl)aminoethyl (meth)acrylate, 2-(diethylamino)ethyl(meth)acrylate, diisopropylaminoethyl (meth)acrylate, mono-t-butylaminoethyl(meth)acrylate and N-morpholinoethyl (meth)acrylate, vinyl aniline, 2- and 4-vinyl pyridine, N-vinyl carbazole, vinyl imidazole, hydroxyethyl(meth)acrylate, hydroxypropyl(meth)acrylate, maleic acid, fumaric acid, itaconic acid and vinyl benzoic acid.

Responsive macromonomers may also be used and include: monomethoxy and monohydroxy poly(propylene oxide) (meth)acrylate and other responsive polymers with terminal functional groups which can be post-functionalised with a polymerisable moiety such as (meth)acrylate, (meth)acrylamide or styrenic groups.

Monomers based on styrene or those containing an aromatic functionality such as styrene, α-methyl styrene, vinyl benzyl chloride, vinyl naphthalene, vinyl benzoic acid, N-vinyl carbazole, 2-, 3- or 4-vinyl pyridine, vinyl aniline, acetoxy styrene, styrene sulfonic acid, vinyl imidazole or derivatives thereof may also be used.

The multifunctional monomer or brancher may comprise a molecule containing at least two vinyl groups which may be polymerised via addition polymerisation. The molecule may be hydrophilic, hydrophobic, amphiphilic, neutral, cationic, zwitterionic, oligomeric or polymeric. Such molecules are often known as cross-linking agents in the art and may be prepared by reacting any di- or multifunctional molecule with a suitably reactive monomer. Examples include: di- or multivinyl esters, di- or multivinyl amides, di- or multivinyl aryl compounds, di- or multivinyl alkyl/aryl ethers. Typically, in the case of oligomeric or polymeric di- or multifunctional branching agents, a linking reaction is used to attach a polymerisable moiety to a di- or multifunctional oligomer or polymer. The brancher may itself have more than one branching point, such as T-shaped divinylic oligomers or polymers. In some cases, more than one multifunctional monomer may be used. When the multifunctional monomer is providing the necessary hydrophilicity in the copolymer, it is preferred that the multifunctional monomer has a molecular weight of at least 1000 Daltons.

The corresponding allyl monomers to those listed above can also be used where appropriate.

Preferred multifunctional monomers or branchers include but are not limited to: divinyl aryl monomers such as divinyl benzene; (meth)acrylate diesters such as ethylene glycol di(meth)acrylate, propyleneglycol di(meth)acrylate and 1,3-butylenedi(meth)acrylate; polyalkylene oxide di(meth)acrylates such as tetraethyleneglycol di(meth)acrylate, poly(ethyleneglycol)di(meth)acrylate and poly(propyleneglycol)di(meth)acrylate; divinyl(meth)acrylamides such as methylene bisacrylamide;

silicone-containing divinyl esters or amides such as (meth)acryloxypropyl-terminated poly(dimethylsiloxane);
divinyl ethers such as poly(ethyleneglycol)divinyl ether; and tetra- or tri-(meth)acrylate esters such as pentaerythritol tetra(meth)acrylate, trimethylolpropane tri(meth)acrylate or glucose di- to penta(meth)acrylate.

Further examples include: vinyl or allyl esters, amides or ethers of pre-formed oligomers or polymers formed via ring-opening polymerisation such as oligo(caprolactam), oligo(caprolactone), poly(caprolactam) or poly(caprolactone), or oligomers or polymers formed via a living polymerisation technique such as oligo- or poly(1,4-butadiene).

Macrocrosslinkers or macrobranchers (multifunctional monomers having a molecular weight of at least 1000 Daltons) are generally formed by linking a polymerisable moiety, such as a vinyl or aryl group, to a pre-formed multifunctional polymer via a suitable linking unit such as an ester, an amide or an ether. Examples of suitable polymers include: di-functional poly(alkylene oxides) such as poly(ethyleneglycol) or poly(propyleneglycol), silicones such as poly(dimethylsiloxane)s, polymers formed by ring-opening polymerisation such as poly(caprolactone) or poly(caprolactam) or poly-functional polymers formed via living polymerisation such as poly(1,4-butadiene).

Preferred macrobranchers include: poly(ethyleneglycol)di(meth)acrylate, poly(propyleneglycol)di(meth)acrylate, methacryloxypropyl-terminated poly(dimethylsiloxane), poly(caprolactone)di(meth)acrylate and poly(caprolactam) di(meth)acrylamide.

Branchers include: methylene bisacrylamide, glycerol di(meth)acrylate, glucose di- and tri(meth)acrylate, oligo(caprolactam) and oligo(caprolactone).

Multi end-functionalised hydrophilic polymers may also be functionalised using a suitable polymerisable moiety such as a (meth)acrylate, (meth)acrylamide or styrenic group.

Further branchers include: divinyl benzene, (meth)acrylate esters such as ethyleneglycol di(meth)acrylate, propyleneglycol di(meth)acrylate and 1,3-butylene di(meth)acrylate, oligo(ethylene glycol)di(meth)acrylates such as tetraethylene glycol di(meth)acrylate, tetra- or tri-(meth)acrylate esters such as pentaerthyritol tetra(meth)acrylate, trimethylolpropane tri(meth)acrylate and glucose penta(meth)acrylate. Multi end-functionalised hydrophobic polymers may also be functionalised using a suitable polymerisable moiety such as a (meth)acrylate, (meth)acrylamide or styrenic group.

Multifunctional responsive polymers may also be functionalised using a suitable polymerisable moiety such as a (meth)acrylate, (meth)acrylamide or styrenic group such as poly(propylene oxide)di(meth)acrylate.

Styrenic branchers, or those containing aromatic functionality are particularly preferred including divinyl benzene, divinyl naphthalene, acrylate or methacrylate derivatives of 1,4 or 1,3 or 1,2 derivatives of dihydroxy dimethyl benzene and derivatives thereof.

### EXAMPLES

The present invention will now be explained in more detail by reference to the following non-limiting examples and drawings wherein:

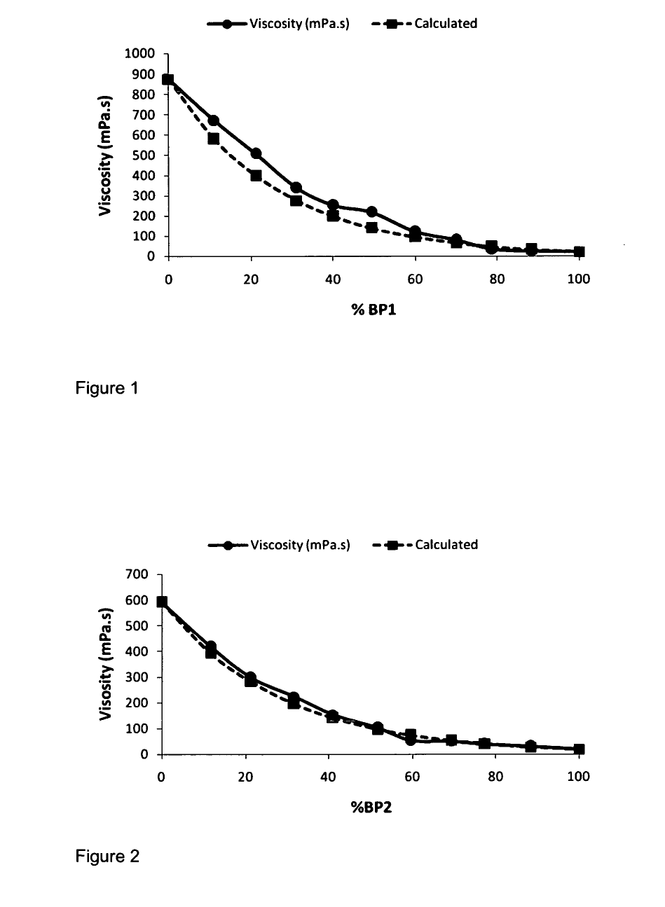

FIG. 1—illustrates the experimental and predicted solution viscosities for a blend of a branched addition polymer (BP1) and a linear polymer (LP5) of varying solution viscosities.

FIG. 2—illustrates the experimental and predicted solution viscosities for a blend of a branched addition polymer (BP2) and a linear polymer (LP1) of varying solution viscosities.

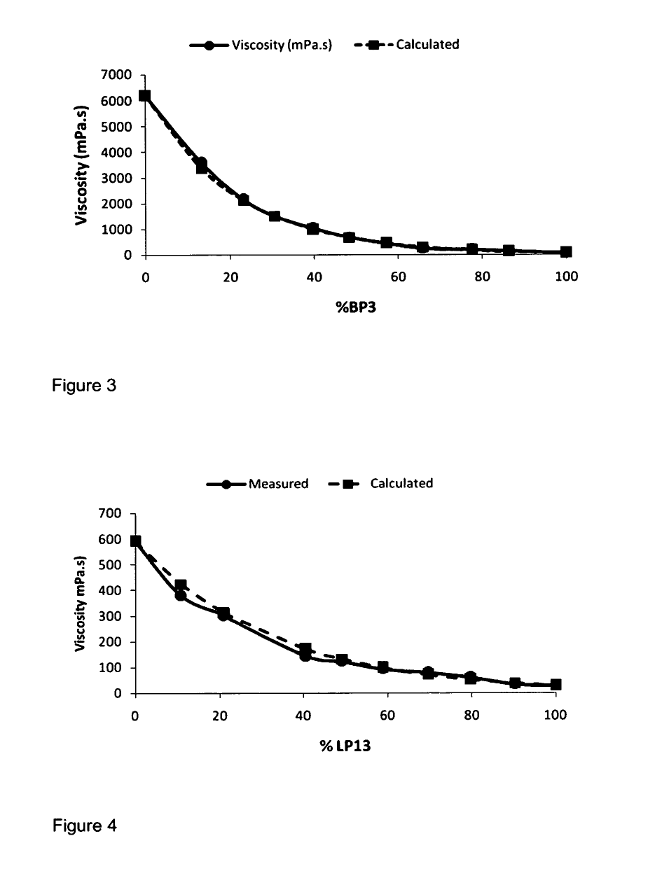

FIG. 3—illustrates the experimental and predicted solution viscosities for a blend of a branched addition polymer (BP3) and a linear polymer (LP2) of varying solution viscosities.

FIG. 4—illustrates the experimental and predicted solution viscosities for a blend of a linear polymer (LP1) and a linear polymer (LP13) of varying solution viscosities.

In addition, in the following examples, copolymers are described using the following nomenclature:

### (MonomerG)_{g} (Monomer J)ⱼ (Brancher L)ₗ (Chain Transfer Agent)_{d}

wherein the values in subscript are the molar ratios of each constituent normalised to give the monofunctional monomer values as 100, that is, g+j=100. The degree of branching or branching level is denoted by I and d refers to the molar ratio of the chain transfer agent.
For example:

Methacrylic acid₁₀₀ Ethyleneglycol dimethacrylate₁₅ Dodecane thiol₁₅ would describe a polymer containing methacrylic acid:ethyleneglycol dimethacrylate:dodecane thiol at a molar ratio of 100:15:15.

### ABBREVIATIONS

### Monomers:

### AA Acrylic Acid

BMA n-Butyl methacrylate
EMA Ethyl methacrylate
HPMA 2-Hydroxypropyl methacrylate
IBMA i-butyl methacrylate
IBOMA Isobornyl methacrylate
MMA Methyl methacrylate

### St Styrene

### Branchers:

DVB divinyl benzene 80%
EGDMA ethyleneglycol dimethacrylate
TEGDMA triethyleneglycol dimethacrylate

### CTA

2ME 2-mercaptoethanol
DDT dodecanethiol
HT hexanethiol

### Initiators

AIBN Azobis isobutyronitrile
DI Luperox DI (Di-t-butyl peroxide)
P Luperox P (t-butyl peroxybenzoate)

### Solvents

### BuOAc Butyl Acetate

### AD 40 Exxsol AD 40

DDT 1-Dodecane thiol

### MEK Butan-2-one

MPA 1-Methoxy 2-propyl acetate

### MeOH Methanol

PE 100-120 Petroleum ether 100-120

### Tol Toluene

All materials were obtained from the Aldrich Chemical Company with the exception of Luperox® DI and P obtained from Arkema Chemical Company, and AD 40 from Exxon Mobil.

### Synthesis and Characterisation

### General Procedure:

Into a three-necked round bottom flask fitted in a DrySyn® Vortex Overhead Stirrer system and equipped with a condenser the required monomers and solvent were introduced. The solution was then degassed for 10 minutes by bubbling nitrogen through it. The solution was then heated to the appropriate temperature and stirred at 320 revolutions per minute (rpm). Once the expected temperature had been reached, the initiator was added and the reaction was allowed to commence and was reacted for at least 4 hours and 30 minutes. When the monomer conversion was found to be greater than 99% (measured by ¹H NMR), the reaction mixture was cooled to room temperature and poured into a vessel. The polymers were characterised by Triple Detection-Size Exclusion Chromatography (TD-SEC).

For example

### BP1

### BMA₁₀₀EGDMA₁₅DDT₁₅

BMA (40 g, 0.281 mol), EGDMA (8.36 g, 42 mmol), DDT (8.54 g, 42 mmol) and petroleum ether (100 to 120° C. fraction) (40 g) were added to a 100 mL flask fitted with an overhead stirred. The solution was degassed for 10 minutes by sparging with nitrogen. The solution was then stirred and heated to 120° C. At reflux, the initiator Luperox® DI (0.710 g, 4.8 mmol) was added. After 18 hours, the reaction mixture was cooled to room temperature. The branched polymer was characterised as follows: Mn 2,200 Da, Mw 340,000 Da, Mw/Mn 138, a 0.414, viscosity 206 mPa·s at 25° C.

### Triple Detection-Size Exclusion Chromatography.

Triple Detection-Size Exclusion Chromatography was performed using a Viscotek instrument and includes a GPC max eluent pump and autosampler, which is coupled to a TDA302 column oven and a multidetector module. The columns used were two ViscoGel HHR-H columns and a guard column with an exclusion limit for polystyrene of 10⁷ g·mol⁻¹.

Tetrahydrofuran (THF) was the mobile phase, and the column oven temperature was set to 35° C., and the flow rate was 1 mL·min⁻¹. The samples were prepared for injection by dissolving 10 mg of polymer in 1.5 mL of HPLC grade THF and then filtered with an Acrodisc® 0.2 μm PTFE membrane. 0.1 mL of this mixture was then injected, and data points were collected for 30 minutes. Omnisec software was used to collect and process the signals transmitted from the detectors to the computer and to calculate the molecular weight.

### Polymer Examples: Linear and Branched.

A series of linear and branched polymers were synthesised according to the general procedure above.

LP1: is a commercially available linear methylmethacylate butylmethyacrylate polymer of molecular weight 50,000
LP2: is a commercially available linear methylmethacylate butylmethyacrylate polymer of molecular weight 200,000
LP5: Polybutyl methacrylate (Aldrich Chemical Company)

### LP6: Polystyrene Aldrich (Aldrich Chemical Company)

LP7: Polyacrylic acid (Acros Chemical Company)

Table 1 describes the synthetic details for preparing the branched copolymers used in accordance with the present invention and linear analogues.

TABLE 1

| | | | Temperature | | |
|---|---|---|---|---|---|
| | | | (degrees | | |
| Example | Solid | | centigrade | | Amount of |
| number | content^{a} | Solvent | ° C.) | Initiator | initiator^{b} |
| BP1 | 60 | PE | 120 | DI | 2.00% |
| | | 100-120 | | | |
| BP2 | 50 | xylene | 145 | DI | 2.00% |
| BP3 | 55 | xylene | 145 | DI | 1.20% |
| BP4 | 55 | xylene | 145 | DI | 1.25% |
| BP5 | 50 | xylene | 70 | AIBN | 2.00% |
| BP6 | 50 | xylene | 145 | DI | 1.20% |
| BP7 | 50 | xylene | 145 | DI | 1.20% |
| BP8 | 50 | xylene | 145 | DI | 1.50% |
| BP9 | 55 | xylene | 145 | DI | 1.50% |
| BP10 | 30 | xylene | 145 | DI | 1.50% |
| BP11 | 35 | MPA | 145 | DI | 1.50% |
| BP12 | 40 | MPA | 145 | DI | 1.50% |
| BP13 | 35 | MPA | 145 | DI | 1.50% |
| BP14 | 42 | MeOH | 70 | AIBN | 0.55% |
| BP15 | 35 | BuOAc | 127 | P | 1.88% |
| BP16 | 35 | BuOAc | 127 | P | 1.88% |
| BP17 | 75 | D40 | 150 | DI | 1.33% |
| BP18 | 75 | D40 | 150 | DI | 1.33% |
| BP19 | 75 | D40 | 150 | DI | 1.33% |
| BP20 | 75 | D40 | 150 | DI | 1.33% |
| BP21 | 55 | xylene | 145 | DI | 2.00% |
| BP22 | 50 | xylene | 145 | DI | 1.5% |
| BP23 | 50 | Xylene | 145 | DI | 1.5 |
| LP3 | 40 | xylene | 145 | DI | 1.50% |
| LP4 | 40 | xylene | 145 | DI | 1.50% |
| LP8 | 30 | BuOAc | 127 | P | 2.25% |
| LP9 | 30 | BuOAc | 127 | P | 2.25% |
| LP10 | 30 | xylene | 145 | DI | 1.50% |
| LP11 | 30 | xylene | 145 | DI | 1.50% |
| LP12 | 30 | xylene | 145 | DI | 1.50% |
| LP13 | 50 | toluene | 85 | AIBN | 2.00% |

Table 1 provides details of the components used for the synthesis of the branched copolymers according to the present invention and the linear polymers compared for comparison. In Table 1:
^{a}is the solid content weight percent (%),
^{b}is the mole percent (Mol. %) relative to the number of double bonds,
c is the total time for the synthesis.

In Table 2 there is provided the compositional and analytical data produced for the branched and linear polymers in Table 1.

TABLE 2
Compositional and analytical data.

| Example | | | | Mw/ | |
|---|---|---|---|---|---|
| number | Composition^{d} | Mn^{e} | Mw^{e} | Mn | α |
| BP1 | BMA₁₀₀EGDMA₁₅DDT₁₅ | 2.2 | 304 | 138.0 | 0.414 |
| BP2 | MMA₆₈BMA₂₈AA₄DVB₁₅DDT₁₅ | 3.0 | 78 | 26.0 | 0.387 |
| BP3 | MMA₆₈BMA₂₈AA₄DVB₁₅DDT₁₅ | 10.6 | 230 | 22.0 | 0.370 |
| BP4 | MMA₅₈BMA₃₈AA₄DVB₁₅DDT₁₅ | 3.8 | 128 | 34.0 | 0.397 |
| BP5 | MMA₄₈BMA₄₈AA₄DVB₁₅DDT₁₅ | 12.3 | 77 | 6.3 | 0.388 |
| BP6 | MMA₇₈BMA₂₂DVB₁₅₂2ME₁₅ | 5.2 | 151 | 29.0 | 0.437 |
| BP7 | MMA₅₀IBOMA₃₅BMA₁₀AA₅DVB₁₅DDT₁₅ | 1.8 | 43 | 24.0 | 0.523 |
| BP8 | MMA₉₈AA₂EGDMA₁₅DDT₁₆ | 3.3 | 306 | 92.0 | 0.601 |
| BP9 | MMA₉₈AA₂EGDMA₅DDT_{6.5} | 30.0 | 622 | 21.0 | 0.533 |
| BP10 | MMA₉₈AA₂EGDMA₁₅DDT₁₆ | 0.9 | 17 | 19.0 | 0.601 |
| BP11 | ST₁₀₀TEGDMA₅DDT_{5.5} | 60.0 | 790 | 13.0 | 0.540 |
| BP12 | ST₁₀₀TEGDMA₁₀DDT₁₅ | 4.7 | 325 | 69.0 | 0.420 |
| BP13 | ST₁₀₀TEGDMA₁₅DDT_{16.5} | 15.7 | 531 | 34.0 | 0.460 |
| BP14 | AA₁₀₀EGDMA₁₀2ME₁₅ | 16.9 | 20.3 | 1.2 | 0.560 |
| BP15 | MMA₄₀BMA₂₀HPMA₄₀EGDMA_{1.33}2ME₂ | 6.6 | 76.8 | 12.0 | 0.497 |
| BP16 | MMA₄₉BMA_{24.5}HPMA_{26.5}EGDMA_{1.23}2ME_{1.85} | 8.15 | 67.5 | 8.3 | 0.522 |
| BP17 | IBMA₉₅AA₅DVB₂₅DDT₂₈ | 4.4 | 56 | 13.0 | 0.367 |
| BP18 | IBMA₉₅AA₅DVB₃₀DDT₃₀ | 2.7 | 143 | 53.0 | 0.391 |
| BP19 | EMA₉₅AA₅DVB₂₅HT₂₈ | 3.0 | 64 | 21.0 | 0.558 |
| BP20 | EMA₉₅AA₅DVB₃₀DDT₃₀ | 6.2 | 498 | 80.0 | 0.410 |
| BP21 | MMA₅₈BMA₃₈AA₄DVB₁₅DDT₁₅ | 7.8 | 189 | 24 | 0.58 |
| BP22 | MMA₉₈AA₂EGDMA₅DDT_{6.5} | 2.5 | 116 | 46 | 0.415 |
| BP23 | MMA₁₀₀DVB₅₂2ME₈ | 2.0 | 22 | 15 | 0.605 |
| LP1 | MMABMA | N/D | 50 | N/D | N/D |
| LP2 | MMABMA | N/D | 200 | N/D | N/D |
| LP3 | MMA₉₈AA₂DDT₂ | 2.3 | 9.3 | 4.0 | 0.570 |
| LP4 | MMA₉₈AA₂ | 10.0 | 102 | 10.0 | 0.694 |
| LP5 | BMA₁₀₀ | N/D | 320 | N/D | N/D |
| LP6 | ST₁₀₀ | N/D | 192 | N/D | N/D |
| LP7 | AA₁₀₀ | N/D | 5 | N/D | N/D |
| LP8 | MMA₅₀BMA_{23.1}HPMA_{26.9} | 3.4 | 43 | 13.0 | 0.802 |
| LP9 | MMA₄₀BMA_{19.4}HPMA_{40.6} | 3.6 | 46 | 13.0 | 0.780 |
| LP10 | IBMA₉₅AA₅ | 1.0 | 42.6 | 43 | 0.833 |
| LP11 | EMA₉₅AA₅ | 1.2 | 52.3 | 44 | 0.766 |
| LP12 | EMA₉₅AA₅DDT_{0.5} | 0.6 | 17 | 28 | 0.791 |
| LP13 | MMA₅₀BMA₅₀DTT₂ | 5.3 | 10.9 | 2 | 0.63 |

In Table 2 the compositional and anlytical data for the linear and branched copolymers synthsised are porvided. Also in Table 2,
^{d}represents the molar composition of the polymers
^{e}the relative quantities in kg/mol;
Mn represents the number average molecular weight in kg/mol;
Mw/Mn represents the polydispersity of the polymers
α - represents the Mark-Houwink aplha value.

### Viscosity Measurements

The solution and melt viscosities of the branched addition copolymers were measured as follows:

### Solution:

The polymers were dissolved in an appropriate solvent and made up to the stated percentage weight/weight solutions and the viscosities of the polymers measured on a Brookfield DV-II+Pro Viscometer, fitted with a CP-40 or CP-52 at 25 or 60° C. The results are shown in Tables 3 and 4.

### Melt:

All polymers were measured using a Bohlin CVO 120 controlled stress rheometer fitted with a CP 4°/40 mm cone. The temperature was set to a determined temperature (Table 5) and the viscosity of the polymer was recorded with increasing shear rate.

The branched and linear polymers were blended at varying levels in xylene (40% w/w). The viscosities of these blends were measured on a Brookfield DV-II+Pro Viscometer, fitted with a CP-40 spindle at 25° C., the results of which are shown in Table 3 below.

In Table 3 there is provided the values for the viscosities versus composition for blends of linear and branched polymers and a blend of two linear polymers.

TABLE 3

| LP1/BP2 | | LP1/LP13 | | LP2/BP3 | | LP5/BP1 | |
|---|---|---|---|---|---|---|---|
| Polymer | | Polymer | | Polymer | | Polymer | |
| Ratio, | Viscosity | Ratio, | Viscosity | Ratio, | Viscosity | Ratio, | Viscosity |
| (w/w) | (mPa · s) | (w/w) | (mPa · s) | (w/w) | (mPa · s) | (w/w) | (mPa · s) |
| 100/0 | 591 | 100/0 | 591 | 100/0 | 6185 | 100/0 | 873 |
| 88.2/11.8 | 418 | 88.6/11.4 | 537 | 86.8/13.2 | 3365 | 88.9/11.1 | 670 |
| 78.7/21.3 | 297 | 79.9/20.1 | 443 | 76.9/23.1 | 2129 | 78.8/21.2 | 507 |
| 68.3/31.7 | 223 | 69.5/30.5 | 340 | 69.4/30.6 | 1509 | 68.9/31.1 | 339 |
| 58.9/41.1 | 154 | 60.1/39.9 | 297 | 60.2/39.8 | 986 | 59.9/40.1 | 252 |
| 48.3/51.7 | 104 | 49.8/50.2 | 245 | 51.6/48.4 | 665 | 50.6/49.4 | 219 |
| 40.5/59.5 | 54 | 40.6/59.4 | 230 | 42.7/57.3 | 441 | 40/60 | 122 |
| 30.6/69.4 | 49 | 31.2/68.8 | 184 | 34.1/65.9 | 297 | 30/70 | 82 |
| 22.5/77.5 | 38 | 21.3/78.7 | 161 | 22.5/77.5 | 174 | 21.3/78.7 | 34 |
| 11.6/88.4 | 30 | 11.7/88.3 | 138 | 13.7/86.3 | 116 | 11.6/88.4 | 25 |
| 0/100 | 18 | 0/100 | 120 | 0/100 | 62 | 0/100 | 22 |

Table 3 demonstrates that a viscosity reduction can be achieved by replacing a linear polymer with a branched polymer additive when compared to the composition comprising the linear polymer alone. In addition, this reduction is greater than that achieved when a low molecular weight linear polymer is blended with a higher molecular weight linear polymer such as in the case of blending LP1 with LP4.

That is, the solution viscosity of the above blends of linear and branched copolymers of similar molecular weight decreases with increasing composition of branched copolymer, or linear polymers of varying molecular weight (Mw), as illustrated in FIGS. 1 to 4. In FIGS. 1 to 4 the dashed lines indicate the predicted solution viscosity for a mixture of two polymers of similar composition with different viscosities as predicted by Equation 1.

In Table 4 there is illustrated the solution viscosities for linear and branched polymers and their blends in toluene, water, butyl acetate and AD-40.

TABLE 4

| | | | | | TEM- | | |
|---|---|---|---|---|---|---|---|
| | | | | | PERA- | CON- | |
| | | | | | TURE | CEN- | |
| LINEAR | | BRANCHED | | | (degrees | TRA- | BLEND |
| POLYMER | | POLYMER | | | centi- | TION | VIS- |
| SAM- | | SAM- | | SOL- | grade | (weight | COSITY |
| PLE | % | PLE | % | VENT | ° C.) | %) | (mPa · s) |
| LP4 | 100 | — | — | Toluene | 25 | 35 | 1328.0 |
| — | — | BP7 | 100 | Toluene | 25 | 35 | 69.6 |
| LP13 | 51.6 | BP7 | 48.4 | Toluene | 25 | 35 | 459.0 |
| LP6 | 100 | — | — | Toluene | 25 | 30 | 693.0 |
| — | — | BP9 | 100 | Toluene | 25 | 30 | 32.3 |
| — | — | BP10 | 100 | Toluene | 25 | 30 | 8.0 |
| — | — | Bp11 | 100 | Toluene | 25 | 30 | 13.7 |
| LP6 | 53.3 | BP9 | 46.7 | Toluene | 25 | 30 | 177.0 |
| LP6 | 55.2 | BP10 | 44.8 | Toluene | 25 | 30 | 107.0 |
| LKP6 | 51.9 | BP11 | 48.1 | Toluene | 25 | 30 | 124.0 |
| LP7 | 100 | — | — | Water | 25 | 40 | 64.3 |
| — | — | BP12 | 100 | Water | 25 | 40 | 47.6 |
| LP7 | 54.4 | BP12 | 45.6 | Water | 25 | 40 | 50.1 |
| LP9 | 100 | — | — | BuOAc | 25 | 40 | 787.0 |
| — | — | BP13 | 100 | BuOAc | 25 | 40 | 489.0 |
| LP8 | 100 | — | — | BuOAc | 25 | 40 | 791.0 |
| — | — | BP14 | 100 | BuOAc | 25 | 40 | 241.0 |
| LP8 | 57.5 | BP14 | 42.5 | BuOAc | 25 | 40 | 519.0 |
| LP10 | 100 | — | — | AD40 | 60 | 50 | 3130.0 |
| LP10 | 52.7 | BP15 | 47.3 | AD40 | 60 | 50 | 243.0 |
| LP10 | 59.4 | BP16 | 40.6 | AD40 | 60 | 50 | 304.0 |

Table 4 demonstrates that the viscosity of a blend of linear and branched polymer is lower than for the linear polymer alone. This is true for a variety of polymer compositions and molecular weights and the effect can be seem in both hydrophilic and hydrophobic solvents.

In Table 5 there is provided the melt viscosities of branched and linear polymers and their blends:

TABLE 5

| | | | | TEMPER- | MELT VISCOSITY | | |
|---|---|---|---|---|---|---|---|
| LINEAR | | BRANCHED | | ATURE | (Pa · s) | | |
| POLYMER | | POLYMER | | (degrees | 10 | | 100 |
| Sample | % | Sample | % | centrigrade) | s⁻¹ | 50 s⁻¹ | s⁻¹ |
| LP1 | 100 | | | 180 | 454 | ND | ND |
| | | BP5 | 100 | 180 | 0.6 | 0.5 | 0.5 |
| | | BP-4 | 100 | 180 | 0.6 | 0.5 | 0.5 |
| | | BP-21 | 100 | 180 | 0.4 | 0.4 | 0.4 |
| LP-1 | 65.3 | BP-5 | 34.8 | 180 | 46.2 | 42.4 | 25 |
| LP-1 | 69.3 | BP-4 | 30.7 | 180 | 63.8 | 56.4 | 30.9 |
| LP-1 | 73 | BP-21 | 27 | 180 | 64.4 | 56.8 | 29.8 |
| LP-3 | 100 | | | 160 | 57 | 57.9 | 53 |
| | | BP-10 | 100 | 160 | 0.3 | 0.3 | 0.3 |
| | | BP-8 | 100 | 160 | 1.9 | 1.5 | 1.5 |
| LP-3 | 53.7 | BP-10 | 46.3 | 160 | 9.4 | 7.6 | 7.3 |
| LP-3 | 59.4 | BP-8 | 40.6 | 160 | 26.8 | ND | 21.8 |
| LP-10 | 100 | | | 160 | 98.4 | 65.1 | 43.7 |
| | | BP-17 | 100 | 160 | 0.2 | 0.2 | 0.2 |
| | | BP-18 | 100 | 160 | 0.4 | 0.4 | 0.4 |
| LP-10 | 55.7 | BP-17 | 44.3 | 160 | 10.7 | 9.3 | 8.9 |
| LP-10 | 83.7 | BP-18 | 16.3 | 160 | 80.0 | 50.1 | 30.0 |
| LP-11 | 100 | | | 180 | 123.7 | ND | 63.4 |
| LP-12 | 100 | | | 160 | 42.7 | 39 | 35.2 |
| | | BP-19 | 100 | 180 | 0.18 | 0.18 | 0.18 |
| | | BP-20 | 100 | 180 | 0.41 | 0.41 | 0.41 |
| LP-11 | 78.6 | BP-19 | 21.4 | 180 | 113.3 | 31.1 | 17.7 |
| LP-11 | 88.1 | BP-20 | 11.9 | 180 | 30.4 | 3.9 | 0.5 |
| LP-12 | 81.4 | BP-19 | 18.6 | 160 | 41.7 | 33.8 | 31.5 |

ND—not determined

The effect of viscosity reduction due to replacement of a linear polymer by a branched equivalent material as shown in Table 5 clearly demonstrates that the melt viscosity of a blend of linear an branched polymer is lower than for the linear material alone.

That is, the solution viscosity of the above blends of linear and branched copolymers of similar molecular weight decreases with increasing composition of branched copolymer, or linear polymers of varying Mw, as illustrated in FIGS. 1 to 4. In FIGS. 1 to 4 the dashed lines indicate the predicted solution viscosity for a mixture of two polymers of similar composition with different viscosities as predicted by Equation 1.

The viscosity of a blend of two miscible polymers of similar composition but with varying viscosities can be predicted using Equation 1. That is, the viscosities of blends of branched and linear polymers can be predicted using the relationship in equation 1 even when the molecular weights of the two polymers are either comparable or when the branched polymer has a larger Mw than the linear polymer counterpart or analogue. This theoretical relationship also holds true for a mixture of linear polymers of varying viscosity and molecular weight where the smaller Mw linear material has a lower solution viscosity than an analogue with a larger Mw. Where linear polymers of similar composition are employed, a lowering in solution viscosity can only be achieved by incorporating a lower molecular weight species into the formulation unlike the situation involving the addition of a branched polymer.

ηBlend=η_{BP}^{α}η_{LP}^{(1-α)} Equation 1.

wherein: equation 1 relates to a theoretical relationship of a blend of two polymers of varying solution viscosities and
α—is the weight fraction of a first branched polymer and
η_{BP} is the viscosity of the branched copolymer solution at the same solids content and
η_{LP} is the viscosity of a linear polymer solution at the same solids content.

### Extensional Rheology

A capillary break up extensional Rheometer (CaBER 1, Thermo Haake) was employed to perform capillary break-up experiments. For each measurement the sample was loaded between the 4 mm plate using a syringe. A fluid circulator bath was used to control of the temperature of the plate at 25° C. The initial sample aspect ratio Λ₀=h₀/2R₀, of 0.5 was defined by initial gap, h₀, of 2 mm and plates diameter, 2R₀, of 4 cm which compensate the low surface tension. All measurements (Table 5) were performed in triplicate with a strike time of 25 ms and a Hencky strain of 1.14 or 1.15. After the step strain was applied, the decay of filament was monitored by a near infra-red laser diode (Omron ZLA-4) with resolution of 10 μm. The Trotan ratio was calculated by dividing the extensional viscosity by the zero shear viscosity of the polymer solution; since the polymer solutions were Newtonian the zero shear viscosities were taken as an average viscosity over a stain rate of 1−100 s⁻¹.

### Rotational Rheology

The solution viscosity measurements of LP4, BP22 and BP6 were performed using an AR2000 cone and plate controlled stress rheometer fitted with an 60 mm 2° anodised cone. Viscosity was measured with increasing shear rate from 1-100 s⁻¹ at 25° C. The solution viscosity measurements of LP4/BP8 was measured on a Bohlin CVO controlled stress cone and plate rheometer, fitted with a CP 2°/55 mm cone, at 25° C.

### Surface Tension

A torsion balance fitted with a Krüss DuNouy ring was used to measure the surface tension of the liquids at room temperature (25° C.). If the surface tension was not measured then it was set to 30 for calculation purposes.

The extensional rheology results for the polymer solutions are shown in Table 6.

TABLE 6

| | | | Surface | | Extensional | | | | |
|---|---|---|---|---|---|---|---|---|---|
| Polymer/ | | Density/ | tension/ | Relaxation | Viscosity/ | Experimental | | Viscosity/ | Trotan |
| blend | Solvent | (g/mL) | (mN/m) | time/s | (mPa · s) | break-up time/s | Concentration % | (mPa · s) | ratio |
| LP4 | MEK | 1.05 | 24.7 | 0.0249 | 1.071 | 0.12 | 26.9 | 154 | 7.0 |
| BP22 | MEK | 0.92 | 27.0 | 0.0062 | 0.46 | 0.026 | 50 | 114 | 4.0 |
| BP8 | MEK | 0.93 | 25.7 | none | 0.30 | 0.01 | 50 | 442 | 2 |
| LP4/BP8 | MEK | N/D | 30 | none | 0.087 | 0.005 | 28.6 | 35.8 | 2.4 |
| (38.6/61.4% | | | | | | | | | |
| w/w) | | | | | | | | | |

N/D—Not determined

1. A method for reducing a viscosity of a solution formulation and/or melt formulation, the method comprising:
combining a branched addition copolymer with a polymer in the solution formulation and/or melt formulation, resulting in a blended solution formulation and/or melt formulation, wherein the viscosity of the blended solution formulation and/or melt formulation is lower than the viscosity of the solution formulation and/or melt formulation comprising the polymer alone,
wherein the branched addition copolymer is obtainable by an addition polymerization process.

2. A method as defined in claim 1, wherein the polymer comprises an analogous linear polymer, and wherein the resulting blended solution formulation and/or melt formulation has a viscosity that is lower than the viscosity of the solution formulation and/or melt formulation comprising an equivalent analogous linear polymer with comparable weight average molecular weight alone.

3. A method as defined in claim 2, wherein the weight average molecular weight of the blend is at least 5% higher than the solution formulation and/or melt formulation comprising the equivalent analogous linear polymer alone.

4. A method as defined in claim 1, wherein the branched addition polymer is between 1 and 99% of the blended solution formulation and/or melt formulation.

5. A method as defined in claim 1, wherein the branched addition polymer is between 1 and 70% of the blended solution formulation and/or melt formulation.

6. A method as defined in claim 1, wherein the branched addition polymer is between 1 and 50% of the blended solution formulation and/or melt formulation.

7. A method as defined in claim 1, wherein the branched addition polymer comprises a weight average molecular weight of 2,000 Da to 1,500,000 Da.

8. A method as defined in claim 1, wherein the branched addition polymer comprises a weight average molecular weight of 5,000 Da to 1,000,000 Da.

9. A method as defined in claim 1, wherein the branched addition polymer comprises a weight average molecular weight of 5,000 Da to 700,000 Da.

10. A method as defined in claim 1, wherein the branched addition copolymer comprises at least two chains which are covalently linked by a bridge other than at their ends,
wherein at least two chains comprise at least one ethyleneically monounsaturated monomer,
wherein the bridge comprises at least one ethyleneically polyunsaturated monomer,
wherein the polymer comprises at least one of a residue of a chain transfer agent and a residue of an initiator, and
wherein a mole ratio of polyunsaturated monomers to monounsaturated monomers is in a range of from 1:100 to 1:4.

11. A method as defined in claim 1, wherein the branched addition copolymer comprises at least two chains which are covalently linked by a bridge other than at their ends;
wherein at least two chains comprise at least one ethyleneically monounsaturated monomer,
wherein the bridge comprises at least one ethyleneically polyunsaturated monomer,
wherein the polymer comprises at least one of a residue of a chain transfer agent and a residue of an initiator,
wherein at least one of the monounsaturated monomers and polyunsaturated monomers and chain transfer agents is a hydrophilic residue,
wherein at least one of one of the monounsaturated monomers and polyunsaturated monomers and chain transfer agents is a hydrophobic residue, and
wherein a mole ratio of polyunsaturated monomers to monounsaturated monomers is in a range of from 1:100 to 1:4.

12. A method as defined in claim 1, wherein the branched addition copolymer comprises less than 1% impurity.

13. A method as defined in claim 1, wherein the branched addition copolymer provides solutions of at least 5% higher solids content with equivalent solution viscosity than a linear polymer equivalent.

14. A method as defined in claim 13, wherein, when the blended solution formulation and/or melt formulation is processed or melted, the branched addition copolymer further provides a reduction to the melt temperature or processing temperature of at least 5% compared to the linear polymer equivalent.

15. A method as defined in claim 1, wherein the branched addition copolymer is combined with the polymer in the solution formulation and/or melt formulation according to the following equation:
ηBlend=η_{BP}^{α}η_{LP}^{(1-α)}
wherein the equation relates to a theoretical relationship of a blend of two polymers of varying solution viscosities and
wherein:
α—is the weight fraction of a first branched polymer,
η_{BP} is the viscosity of a branched addition copolymer solution at the same solids content,
η_{LP} is the viscosity of a linear polymer solution at the same solids content, and
η_{Blend} is the measured viscosity of the blended solution formulation and/or melt formulation.

16. A method as defined in claim 1 to reduce the viscosity of the solution formulation and/or melt formulation in application areas selected from the group comprising:
coatings, inks, adhesives, lubricants, composites, oil field recovery agents, metal working fluids, coolants, sealants, films, resins, textiles, injection mouldings, water treatment, electronics, cosmetics, pharmaceuticals, agrochemicals and lithography.

17. A polymer blend, comprising:
a branched addition copolymer as defined in claim 1, wherein a copolymer is obtainable by an addition polymerization process, wherein the branched addition copolymer comprises a weight average molecular weight of 2,000 Da to 1,500,000 Da, and wherein the branched addition copolymer comprises:
at least two chains which are covalently linked by a bridge other than at their ends,
wherein at least two chains comprise at least one ethyleneically monounsaturated monomer,
wherein the bridge comprises at least one ethyleneically polyunsaturated monomer,
wherein the polymer comprises at least one of a residue of a chain transfer agent and a residue of an initiator,
wherein at least one of the monounsaturated monomers and polyunsaturated monomers and chain transfer agents is a hydrophilic residue,
wherein at least one of one of the monounsaturated monomers and polyunsaturated monomers and chain transfer agents is a hydrophobic residue, and
wherein a mole ratio of polyunsaturated monomers to monounsaturated monomers is in a range of from 1:100 to 1:4,
the polymer blend further comprising a linear equivalent polymer,
wherein the polymer blend comprises between 10 and 90% branched addition copolymer and between 10 and 90% linear equivalent polymer.

18. A polymer blend as defined in claim 17, wherein the monomers used to prepare the branched addition copolymers are vinylic or allylic and are selected from the group comprising:
styrenics, acrylics, methacrylics, allylics, acrylamides, methacrylamides, vinyl or allyl acetates, N-vinyl or allyl amines and vinyl or allyl ethers.

19. A method as defined in claim 1, wherein the branched addition copolymer comprises units selected from the group consisting of:
styrene, vinyl benzyl chloride, 2-vinyl pyridine, 4-vinyl pyridine, methyl acrylate, ethyl acrylate, methyl methacrylate, ethyl methacrylate, butyl methacrylate, butyl acrylate, acrylic acid, methacrylic acid, 2-hydroxyethyl methacrylate, 2-hydroxy ethyl acrylate, 2-hydroxypropyl acrylate, 2-hydroxypropyl methacrylate, acrylamide, methacrylamide, dimethyl acrylamide, dimethyl(meth)acrylamide, allyl methacrylate, dimethylaminoethyl methacrylate, dimethylaminoethyl acrylate, diethylaminoethyl methacrylate, diethylaminoethyl acrylate, divinyl benzene, ethyleneglycol dimethacrylate, ethyleneglycol di acrylate, triethylene glycol dimethacrylate, tetraethyleneglycol dimethacrylate, tetraethyleneglycol diacrylate, 1,3,5-triallyl-1,3,5-triazine-2,4,6(1H,3H,5H)-trione, dodecane thiol, hexane thiol, 2-mercaptoethanol and fragments arising from azobis isobutyronitrile, di-t-butyl peroxide and t-butyl peroxybenzoate.

20. A method as defined in claim 1, wherein the branched addition copolymer comprises units selected from the group consisting of:
styrene, 2-vinyl pyridine, 4-vinyl pyridine, methyl acrylate, methyl methacrylate, butyl methacrylate, butyl acrylate, acrylic acid, methacrylic acid, acrylamide, methacrylamide, dimethyl acrylamide, dimethyl(meth)acrylamide, divinyl benzene, ethyleneglycol dimethacrylate, ethyleneglycol diacrylate, triethylene glycol dimethacrylate, dodecane thiol, hexane thiol, 2-mercaptoethanol, azobis isobutyronitrile, di-t-butyl peroxide and t-butyl peroxybenzoate.

21. A formulation, comprising:
a branched addition copolymer as defined in claim 1 in combination with a linear equivalent polymer as defined in claim 17 and a liquid medium,
wherein the liquid medium comprises at least one of an organic solvent and an aqueous solvent.

22. A formulation as defined in claim 21,
wherein the formulation is used to reduce the viscosity of a solution formulation or melt formulation comprising an equivalent linear polymer by at least 20%.
